(19) United States
(12) Patent Application Publication
(10) Pub. No.: US 2023/0391961 A1
(43) Pub. Date: Dec. 7, 2023

(54) METHODS FOR INSERTING NANOPORES INTO POLYMERIC MEMBRANES USING CHAOTROPIC SOLVENTS

(71) Applicant: Illumina Cambridge Limited, Cambridge (GB)

(72) Inventors: Istvan Kocsis, Cambridge (GB); Charlotte Vacogne, Cambridge (GB); Oliver Uttley, Cambridge (GB); Antonio Conde-Gonzalez, Cambridge (GB); Alexandre Richez, Cambridge (GB)

(73) Assignee: Illumina Cambridge Limited, Cambridge (GB)

(21) Appl. No.: 18/193,512

(22) Filed: Mar. 30, 2023

Related U.S. Application Data
(60) Provisional application No. 63/325,735, filed on Mar. 31, 2022.

(51) Int. Cl. C08G 81/00; C12Q 1/6869

(52) U.S. Cl. CPC C08G 81/00; C12Q 1/6869

(57) ABSTRACT

Methods of inserting a nanopore into a polymeric membrane are provided herein. The membrane may be destabilized using a chaotropic solvent. The nanopore may be inserted into the destabilized polymer membrane. The chaotropic solvent may be removed to stabilize the polymer membrane with the nanopore inserted therein.

### CROSS-REFERENCE TO RELATED APPLICATIONS

This application claims the benefit of U.S. Provisional Patent Application No. 63/325,735, filed on Mar. 31, 2022 and entitled “METHODS FOR INSERTING NANOPORES INTO POLYMERIC MEMBRANES USING CHAOTROPIC SOLVENTS”, the entire contents of which are incorporated by reference herein.

### FIELD

This application relates to the insertion of nanopores into polymeric membranes.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein nanopore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Methods for inserting nanopores into polymeric membranes using chaotropic solvents are provided herein.

Some examples herein provide a method of inserting a nanopore into a polymer membrane. The method may include destabilizing the polymer membrane using a chaotropic solvent, inserting the nanopore into the destabilized polymer membrane, and removing the chaotropic solvent to stabilize the polymer membrane with the nanopore inserted therein.

In some examples, the chaotropic solvent includes an amphiphilic solvent. In some examples, the amphiphilic solvent includes an alcohol, tetrahydrofuran, acetaldehyde, acetic acid, acetone, acetonitrile, ethylamine, or propanoic acid. In some examples, the alcohol includes isopropanol, n-butanol, ethanol, methanol, or 1-propanol. In some examples, the amphiphilic solvent includes a carbon chain with a length between 1 and 6 carbons. In some examples, the amphiphilic solvent has a molar mass of less than about 75 grams per mole.

In some examples, the chaotropic solvent includes a highly polar solvent. In some examples, the highly polar solvent includes a carbonyl group or a sulfonyl group. In some examples, the highly polar solvent has a molar mass of less than about 80 grams per mole. In some examples, the highly polar solvent includes dimethyl sulfoxide, acetyl cyanide, urea, acetonitrile, formamide, dimethylformamide, methyl isocyanide, N-methyl-2-pyrrolidone, or triethylene glycol.

In some examples, the chaotropic solvent is removed through repeated dilutions using a buffer solution. In some examples, the chaotropic solvent is removed through diffusion out of the polymer membrane.

In some examples, the nanopore is inserted into the destabilized polymer membrane using electroporation, pipette pump cycle, or detergent assisted nanopore insertion.

In some examples, the polymer membrane includes molecules of a diblock copolymer, the molecules of the diblock copolymer including a hydrophobic block and a hydrophilic block coupled to the hydrophobic block. In some examples, the polymer membrane includes a first layer including a first plurality of molecules of the diblock copolymer, and a second layer including a second plurality of molecules of the diblock copolymer. The hydrophilic blocks of the first plurality of molecules may form a first outer surface of the polymer membrane. The hydrophilic blocks of the second plurality of molecules may form a second outer surface of the polymer membrane. The hydrophobic blocks of the first and second pluralities of molecules may contact one another within the polymer membrane. In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, the polymer membrane includes molecules of a triblock copolymer. In some examples, each molecule of the triblock copolymer includes a hydrophilic block and first and second hydrophobic blocks, the hydrophilic block being coupled to and between the first and second hydrophobic blocks. In some examples, the polymer membrane includes a first layer including a first plurality of molecules of the triblock copolymer and a second layer including a second plurality of molecules of the triblock copolymer. The hydrophilic blocks of the first plurality of molecules may form a first outer surface of the polymer membrane. The hydrophilic blocks of the second plurality of molecules may form a second outer surface of the polymer membrane. The hydrophobic blocks of the first and second pluralities of molecules may contact one another within the polymer membrane. In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, each molecule of the triblock copolymer includes a hydrophobic block and first and second hydrophilic blocks, the hydrophobic block being coupled to and between the first and second hydrophilic blocks. In some examples, the polymer membrane comprises at least one layer including a plurality of molecules of the triblock copolymer, the first hydrophilic blocks and the second hydrophilic blocks of the second plurality of molecules forming first and second outer surfaces of the polymer membrane.

In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, the chaotropic solvent is neither an amphiphilic solvent nor a highly polar solvent, and is used a sufficiently high concentration to destabilize the membrane.

In some examples, the nanopore includes α-hemolysin. In some examples, the nanopore includes MspA.

Some examples herein provide a composition. The composition may include a polymer membrane, and a chaotropic solvent destabilizing the polymer membrane.

In some examples, the chaotropic solvent includes an amphiphilic solvent. In some examples, the amphiphilic solvent includes an alcohol, tetrahydrofuran, acetaldehyde, acetic acid, acetone, acetonitrile, ethylamine, or propanoic acid. In some examples, the alcohol includes isopropanol, n-butanol, ethanol, methanol, or 1-propanol. In some examples, the amphiphilic solvent includes a carbon chain with a length between 1 and 6 carbons. In some examples, the amphiphilic solvent has a molar mass of less than about 75 grams per mole.

In some examples, the chaotropic solvent includes a highly polar solvent. In some examples, the highly polar solvent includes a carbonyl group or a sulfonyl group. In some examples, the highly polar solvent has a molar mass of less than about 80 grams per mole. In some examples, the highly polar solvent is dimethyl sulfoxide, acetyl cyanide, urea, acetonitrile, formamide, dimethylformamide, methyl isocyanide, N-methyl-2-pyrrolidone, or triethylene glycol.

In some examples, the polymer membrane includes molecules of a diblock copolymer, the molecules of the diblock copolymer including a hydrophobic block and a hydrophilic block coupled to the hydrophobic block. In some examples, the polymer membrane includes a first layer including a first plurality of molecules of the diblock copolymer, and a second layer including a second plurality of molecules of the diblock copolymer. The hydrophilic blocks of the first plurality of molecules may form a first outer surface of the polymer membrane. The hydrophilic blocks of the second plurality of molecules may form a second outer surface of the polymer membrane. The hydrophobic blocks of the first and second pluralities of molecules may contact one another within the polymer membrane.

In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, the polymer membrane includes molecules of a triblock copolymer. In some examples, each molecule of the triblock copolymer may include a hydrophilic block and first and second hydrophobic blocks, the hydrophilic block being coupled to and between the first and second hydrophobic blocks. In some examples, the polymer membrane includes a first layer including a first plurality of molecules of the triblock copolymer and a second layer including a second plurality of molecules of the triblock copolymer. The hydrophilic blocks of the first plurality of molecules may form a first outer surface of the membrane. The hydrophilic blocks of the second plurality of molecules may form a second outer surface of the membrane. The hydrophobic blocks of the first and second pluralities of molecules may contact one another within the membrane. In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, each molecule of the triblock copolymer may include a hydrophobic block and first and second hydrophilic blocks, the hydrophobic block being coupled to and between the first and second hydrophilic blocks. In some examples, the polymer membrane includes at least one layer comprising a plurality of molecules of the triblock copolymer, the first hydrophilic blocks and the second hydrophilic blocks of the second plurality of molecules forming first and second outer surfaces of the polymer membrane. In some examples, the chaotropic solvent destabilizes the polymer membrane by intercalating between the hydrophilic blocks. In some examples, the chaotropic solvent destabilizes the polymer membrane by intercalating at interfaces between the hydrophilic blocks and the hydrophobic blocks.

In some examples, the chaotropic solvent is neither an amphiphilic solvent nor a highly polar solvent, and is used a sufficiently high concentration to destabilize the membrane.

In some examples, the composition further includes a nanopore within the polymer membrane. In some examples, the nanopore includes α-hemolysin. In some examples, the nanopore includes MspA.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a polymeric membrane.

FIGS. 2A-2B schematically illustrate plan and cross-sectional views of further details of the nanopore composition and device of FIG. 1.

FIGS. 3A-3D schematically illustrate operations for inserting a nanopore into an example polymeric membrane using a first type of chaotropic solvent.

FIG. 4 schematically illustrates the operation described with reference to FIG. 3C, using a different type of chaotropic solvent.

FIGS. 5A-5C schematically illustrate operations for inserting a nanopore into another example polymeric membrane using different types of chaotropic solvents.

FIGS. 6A-6C schematically illustrate operations for inserting a nanopore into another example polymeric membrane using different types of chaotropic solvents.

FIGS. 7A-7C schematically illustrate further details of membranes using block copolymers which may be included in the nanopore composition and device of FIG. 1 and used in respective operations described with reference to FIGS. 3A-6C.

FIG. 8 illustrates an example flow of operations in a method for inserting a nanopore into a polymeric membrane.

FIG. 9 illustrates the voltage breakdown waveform used to assess polymeric membrane stability.

FIG. 10 is a plot showing the measured membrane stability of example suspended copolymeric membranes generated using buffer solutions with different concentrations of a chaotropic solvent.

FIG. 11 is a plot showing the number of nanopores inserted into example membranes after washing in chaotropic solvent-containing buffer solutions.

FIG. 12 is a plot showing the number of nanopores inserted into example membranes after washing in different chaotropic solvent-containing buffer solutions.

FIG. 13 schematically illustrates a cross-sectional view of an example use of the composition and device of FIG. 1.

FIG. 14 schematically illustrates a cross-sectional view of another example use of the composition and device of FIG. 1.

FIG. 15 schematically illustrates a cross-sectional view of another example use of the composition and device of FIG. 1.

FIG. 16 schematically illustrates a cross-sectional view of another example use of the composition and device of FIG. 1.

FIG. 17 schematically illustrates a cross-sectional view of another example use of the composition and device of FIG. 1.

### DETAILED DESCRIPTION

Methods for inserting nanopores into polymeric membranes using chaotropic solvents are provided herein.

For example, nanopore sequencing may utilize a nanopore that is inserted into a polymeric membrane (barrier), and that includes an aperture through which ions and/or other molecules may flow from one side of the membrane to the other. Circuitry may be used to detect a sequence, for example of nucleotides. For example, during sequencing-by-synthesis (SBS), on a first side of the membrane, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the membrane, for example to provide suitable ion transport for detection on one side of the membrane, while suitably promoting activity of the polymerase on the other side of the membrane. Accordingly, membrane stability is beneficial. However, it may be difficult to insert nanopores into membranes that are too strong.

As provided herein, a water miscible chaotropic solvent may be used to reversibly destabilize a polymeric membrane for nanopore insertion. As explained in greater detail below, in some examples, the chaotropic solvent may be used to destabilize the polymeric membrane for a period of time during which the nanopore is inserted. The chaotropic solvent then may be removed so as to stabilize the membrane, e.g., so that the membrane has about the same stability as it would under normal conditions (e.g., without destabilization). In some examples, the membrane is sufficiently strong and stable that it may not be possible to insert a nanopore into the membrane without the use of the chaotropic solvent to temporarily destabilize the membrane. Accordingly, after the membrane, with the nanopore therein, is stabilized, the membrane may be expected to be sufficiently strong and stable for prolonged use under forces such as may be applied during use of a device including such a membrane, illustratively genomic sequencing.

First, some terms used herein will be briefly explained. Then, some example methods for inserting a nanopore into a polymeric membrane using a chaotropic solvent, and intermediate structures formed using such methods, will be described.

Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NSSB protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a membrane, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions and water-soluble molecules such as nucleotides or amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a membrane material that is not of biological origin (e.g., polymeric materials, synthetic phospholipids, solid-state membranes, or combinations thereof).

As used herein, a “polymeric membrane” or “polymer membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “polymer membrane,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of the polymeric membrane. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer as one another, and the second block may include a different type of monomer. In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicities and hydrophobicities of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, to “destabilize” a polymeric membrane is intended to mean to generate an arrangement of polymer chains that is substantially disrupted as compared to the arrangement such polymer chains would obtain in a stable membrane. A destabilized polymeric membrane may have a free energy that is substantially higher than that of the same membrane in the stable state. A membrane initially may be generated in a stable state and then destabilized. Alternatively, a membrane initially may be generated in a destabilized state. To “stabilize” a destabilized polymeric membrane is intended to mean to cause the polymer chains of the destabilized membrane to obtain a stable arrangement. As such, a polymeric membrane that is stabilized after being destabilized may have a similar free energy as a membrane which had never been destabilized (which in some examples may be the membrane prior to destabilization), and may have a lower free energy than that membrane in the destabilized state.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic. As another example, the term “amphiphilic solvent” means a solvent that includes a hydrophobic portion and a hydrophilic portion.

As used herein, a “solution” is intended to refer to a homogeneous mixture including two or more substances. In such a mixture, a solute is a substance which is uniformly dissolved in another substance referred to as a solvent. A solution may include a single solute, or may include a plurality of solutes. Additionally, or alternatively, a solution may include a single solvent, or may include a plurality of solvents. An “aqueous solution” refers to a solution in which the solvent is, or includes, water.

A first liquid that forms a homogeneous mixture with a second liquid is referred to herein as being “miscible” or “soluble” with the second liquid.

As used herein, the term “chaotropic solvent” means a water-soluble solvent capable of intercalating between the polymer chains of a polymeric membrane in such a manner as to destabilize the membrane. A chaotropic solvent may destabilize the membrane, for example, by weakening intermolecular interactions between polymer chains, and thereby changing the fluidity and packing density of the polymer chains. The intermolecular interactions which the chaotropic solvent weakens may include any suitable hydrogen bonding, van der Waals interactions, polar interactions, and ionic interactions, depending on the identity of the block copolymer. Chaotropic solvents typically have a molar mass of less than about 150 grams per mole. Examples of chaotropic solvents include amphiphilic solvents, highly polar solvents, and solvents that may not necessarily be either an amphiphilic solvent or a highly polar solvent, but are used a sufficiently high concentration to destabilize a membrane. Amphiphilic solvents may intercalate between polymer chains to a region that is approximately at the interface between hydrophilic and hydrophobic portions of those chains. Highly polar solvents may intercalate between polymer chains to a region that is approximately at the hydrophilic portions of those chains. Solvents that are present at a sufficiently high concentration to destabilize a membrane may intercalate between polymer chains to any suitable region within the membrane.

As used herein, the term “highly polar solvent” means a solvent with an electric dipole moment larger than about 3.5 D. In some examples, a highly polar solvent may include a carbonyl group or a sulfonyl group. Additionally, or alternatively, in some examples the highly polar solvent may have a molar mass of less than about 80 grams per mole. Nonlimiting examples of highly polar solvents include dimethyl sulfoxide, acetyl cyanide, urea, acetonitrile, formamide, dimethylformamide, methyl isocyanide, N-methyl-2-pyrrolidone, and triethylene glycol.

As used herein, the term “amphiphilic solvent” means a solvent that includes a hydrophobic portion and a hydrophilic portion. Alcohols, tetrahydrofuran, acetaldehyde, acetic acid, acetone, acetonitrile, ethylamine, and propanoic acid are nonlimiting examples of amphiphilic solvents. In some examples, the amphiphilic solvent includes a carbon chain with a length between about 1 carbon and about 6 carbons. Additionally, or alternatively, in some examples the amphiphilic solvent has a molar mass of less than about 75 grams per mole. Nonlimiting examples of such alcohols include isopropanol, n-butanol, ethanol, methanol, and 1-propanol.

In some examples, an amphiphilic solvent is characterized by an octanol-water partitioning coefficient (“logP”) between about −2 and about 2, e.g., between about −1.5 and about 1.5, or between about −1 and about 1. As used herein, the term “octanol-water partitioning coefficient,” as applied to a particular species, means the common logarithm of the ratio with the concentration of the species in the octanol-rich phase of an octanol-water experiment in the numerator and the concentration of the species in the water-rich phase of an octanol-water experiment in the denominator. Hydrophilic species have negative octanol-water partitioning coefficients while hydrophobic species have positive octanol-water partitioning coefficients. As used herein, the term “octanol-water experiment” means the introduction of a species into a two-phase system consisting of n-octanol and water.

Other water-soluble solvents, when used at sufficiently high concentrations, may be used to intercalate between the polymer chains of a polymeric membrane in such a manner as to destabilize the membrane (that is, may be used as a chaotropic solvent). For example, an aqueous solution (such as a buffer solution) may be prepared that contains a sufficiently high concentration of the water-soluble solvent to destabilize a membrane, and the buffer solution may be brought into contact with a previously formed membrane so as to destabilize that membrane. Such a use of such water-soluble solvent as a chaotropic solvent may be distinguished from any use of such a solvent while forming the membrane by its timing (being applied to the formed membrane as a chaotropic solvent, as opposed to being used to form the membrane as an organic solvent). Additionally, such a use of such water-soluble solvent as a chaotropic solvent may be distinguished from any use of such a solvent while forming the membrane by the manner in which it is applied (being applied while dissolved in an aqueous solution as a chaotropic solvent, as opposed to being applied while dissolved in, or as, an organic solvent to form the membrane).

As a general rule, solvents with a higher octanol-water partitioning coefficient (logP) may be expected to be useful as chaotropic solvents at a lower concentration, because they would distribute more readily into the bilayer where they can disrupt it. For example, as shown in the working examples herein, n-BuOH may destabilize a membrane at a concentration of about 1 w %, while IPA may be applied at a concentration of about 20% to have similar destabilizing effects. In some examples, solvents with logP values between −2 and 0.5 may be used as chaotropic solvents at concentrations of about 1-20w % in an aqueous solution, and solvents with logP values between 0.5 and 2 may be used as chaotropic solvents at concentrations of about 0.01-1w % in an aqueous solution. It will be appreciated that these ranges are meant only to be examples, and that the particular range may vary based on the particular solvent and the type of membrane the solvent is being used to destabilize.

Non-limiting examples of chaotropic solvents that may not necessarily be either an amphiphilic solvent or a highly polar solvent, but may be used a sufficiently high concentration to destabilize a membrane, are listed in Table 1 below.

TABLE 1

| Name | Chemical formula |
|---|---|
| 1,2-Butanediol | CH₃CH₂CH(OH)CH₂OH |
| 1,3-Butanediol | CH₃CH(OH)CH₂CH₂OH |
| 1,4-Butanediol | HOCH₂CH₂CH₂CH₂OH |
| 2-Butoxyethanol | C₆H₁₄O₂ |
| butyric acid | CH₃CH₂CH₂COOH |
| diethanolamine | HN(CH₂CH₂OH)₂ |
| diethylenetriamine | HN(CH₂CH₂NH₂)₂ |
| dimethoxyethane | C₄H₁₀O₂ |
| 1,4-Dioxane | C₄H₈O₂ |
| ethylene glycol | C₂H₆O₂ |
| formic acid | HCOOH |
| furfuryl alcohol | C₅H₆O₂ |
| glycerol | C₃H₈O₃ |
| methyl diethanolamine | CH₃N(C₂H₄OH)₂ |
| 1,3-Propanediol | CH₂(CH₂OH)₂ |
| 1,5-Pentanediol | HOCH₂CH₂CH₂CH₂CH₂OH |
| 2-Propanol | (CH₃)₂CHOH |
| propylene glycol | HOCH₂CHOHCH₃ |
| pyridine | C₅H₅N |

As used herein, the term “electroporation” means the application of a voltage across a membrane such that a nanopore is inserted into the membrane.

As used herein, the term “linker” is intended to mean a moiety, molecule, or molecules via which one element is attached to another element. Linkers may be covalent, or may be non-covalent. Nonlimiting examples of covalent linkers include moieties such as alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “barrier support” is intended to refer to a structure that can suspend a barrier. When the barrier comprises a membrane (such as a polymeric membrane), the barrier support may be referred to as a “membrane support.” A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

Methods of Inserting Nanopores into Polymeric Membranes Using Chaotropic Solvents

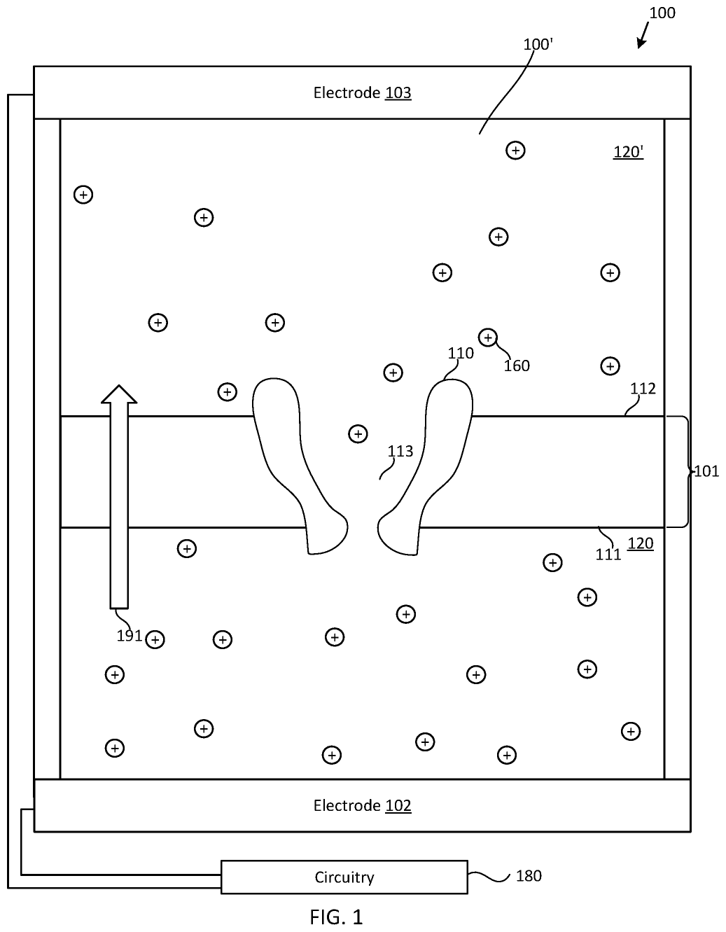

Compositions and systems including nanopores inserted into polymeric membranes, and methods for inserting nanopores into polymeric membranes using chaotropic solvents, now will be described with reference to FIGS. 1-8.

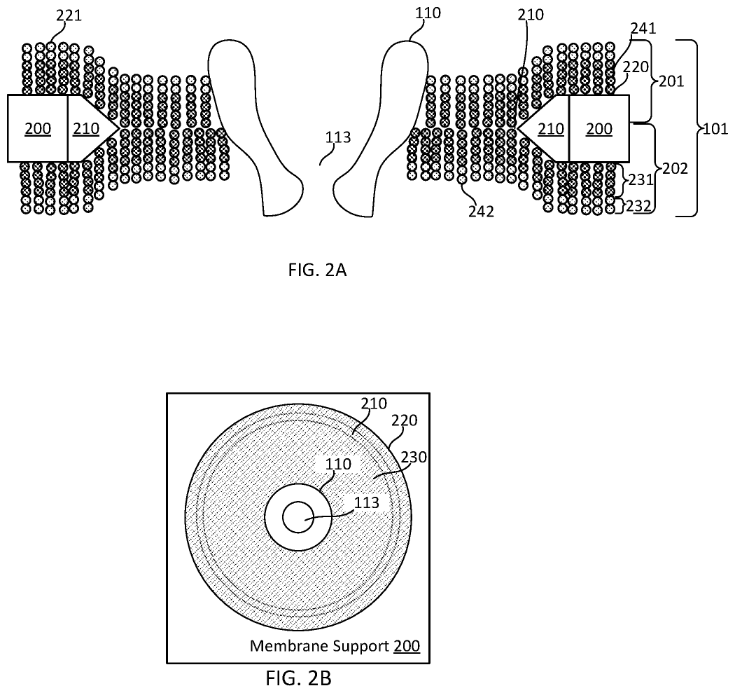

FIG. 1 schematically illustrates a cross-sectional view of an example nanopore composition and device 100 including a polymeric membrane (barrier). Device 100 includes fluidic well 100′ including polymeric membrane 101 having first (trans) side 111 and second (cis) side 112, first fluid 120 within fluidic well 100′ and in contact with first side 111 of the membrane, and second fluid 120′ within the fluidic well and in contact with the second side 112 of the membrane. Polymeric membrane 101 may have any suitable structure that normally inhibits passage of molecules from one side of the membrane to the other side of the membrane, e.g., that normally inhibits contact between fluid 120 and fluid 120′. Illustratively, polymeric membrane 101 may include a diblock or triblock copolymer and may have a structure such as described in greater detail below with reference to FIGS. 2A-2B, 3A-3D, 4, 5A-5C, 6A-6C, or 7A-7C. Illustratively, barrier 101 may include a bilayer including first and second which respectively may be formed using AB diblock copolymers provided herein, or BAB triblock copolymers provided herein, or certain ABA triblock copolymers provided herein, and may have a structure such as described in greater detail below. Alternatively, barrier 101 may include only a single layer, which inhibits the flow of molecules across that layer. Illustratively, barrier 101 may include a single layer which may be formed using certain ABA triblock copolymers provided herein, and may have a structure such as described in greater detail below. In other examples, barrier 101 may be partially a single layer, and partially a bilayer, formed using certain ABA triblock copolymers provided herein, and may have a structure such as described in greater detail below.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water-soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). It will also be appreciated that the first and second fluids optionally may include any suitable combination of other solutes. Illustratively, first and second fluids 120, 120′ may include an aqueous buffer (such as N-(2-hydroxyethyl)piperazine-N′-2-ethanesulfonic acid (HEPES), commercially available from Fisher BioReagents).

Still referring to FIG. 1 device 100 further may include nanopore disposed within membrane 101 and providing aperture 113 fluidically coupling first side 111 to second side 112. As such, aperture 113 of nanopore 110 may provide a pathway for fluid 120 and/or fluid 120′ (e.g., salt 160) to flow through membrane 101. Nanopore 110 may include a solid-state nanopore, a biological nanopore (e.g., MspA such as illustrated in FIG. 1), or a biological and solid-state hybrid nanopore. Nonlimiting examples and properties of membranes and nanopores are described elsewhere herein, as well as in U.S. Pat. No. 9,708,655, the entire contents of which are incorporated by reference herein. In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for sequencing, such as polynucleotide sequencing.

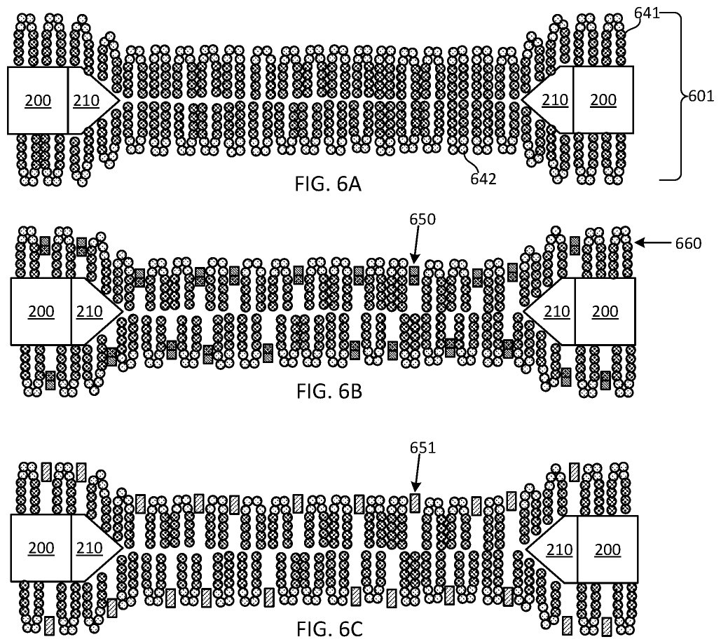

In some examples, polymeric membrane 101 between first and second fluids 120, 120′ includes a block copolymer. For example, FIGS. 2A-2B schematically illustrate plan and cross-sectional views of further details of the nanopore composition and device of FIG. 1. As illustrated in FIG. 2A, membrane (barrier) 101 may include first layer 201 including a first plurality of block copolymer molecules 221 and second layer 202 including a second plurality of the block copolymer molecules. In the nonlimiting example illustrated in FIG. 2A, the copolymer is a diblock copolymer (AB), such that each molecule 221 includes a hydrophobic “B” block 231 (within which circles 241 with darker fill represent hydrophobic monomers) and a hydrophilic “A” block 232 (within which circles 242 with lighter fill represent hydrophilic monomers) coupled directly or indirectly thereto. In other examples such as will be described with reference to FIGS. 5A-5C and 6A-6C, the copolymer instead may include a triblock copolymer (e.g., ABA or BAB, respectively). In the example illustrated in FIG. 2A, the hydrophilic blocks 232 of the first plurality of molecules 221 may form a first outer surface of membrane 101, e.g., the surface of membrane 101 contacting fluid 120 on first side 111. The hydrophilic blocks 232 of the second plurality of molecules 221 may form a second outer surface of membrane 101, e.g., the surface of membrane 101 contacting fluid 120′ on second side 112. The hydrophobic blocks 231 of the first and second pluralities of molecules 221 may contact one another within the membrane.

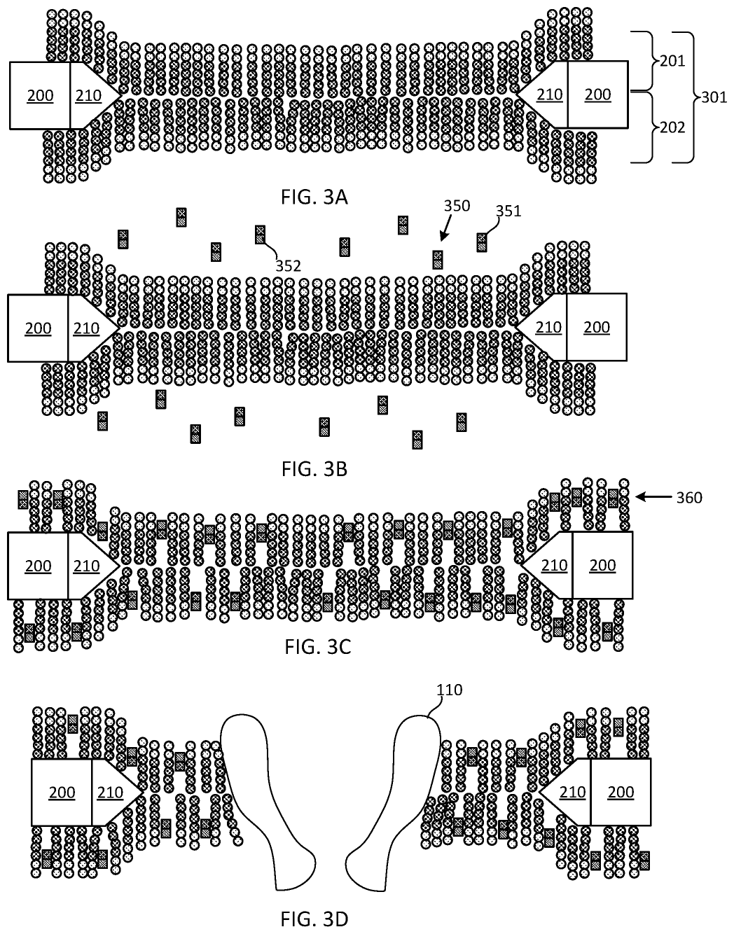

In the example illustrated in FIGS. 2A-2B, membrane 101 may be suspended using a barrier support, e.g., membrane support 200. For example, membrane support 200 may include a substrate having an aperture 230 defined therethrough, e.g., a substantially circular aperture, or an aperture having another shape. Additionally, or alternatively, the barrier support may include one or more features of a well in which the nanopore device is formed, such as a lip or ledge on either side of the well. Nonlimiting examples of materials which may be included in a barrier support are provided further above. An annulus 210 including hydrophobic (non-polar) solvent, and which also may include polymer chains and/or other compound(s), may adhere to membrane support 200 and may support a portion of membrane 101, e.g., may be located within membrane 101 (here, between layer 201 and layer 202). Additionally, annulus 210 may taper inwards in a manner such as illustrated in FIG. 2A. An outer portion of the molecules 221 of membrane 101 may be disposed on support 200 (e.g., the portion extending between aperture 230 and membrane periphery 220), while an inner portion of the molecules may form a freestanding portion of membrane 101 (e.g., the portion within aperture 210, a part of which is supported by annulus 210). Nanopore 110 may be inserted into the freestanding portion of membrane 101 using a chaotropic solvent, e.g., in a manner such as now will be described with reference to FIGS. 3A-3D, 4, 5A-5C, 6A-6C, 7A-7C, and 8.

FIGS. 3A-3D schematically illustrates operations for inserting a nanopore into a polymeric membrane using a first type of chaotropic solvent. FIG. 3A illustrates stable diblock copolymer membrane 301. As illustrated in FIG. 3A, diblock copolymer membrane 301 may be configured similarly as membrane 101 described as reference to FIGS. 2A-2B, e.g., may include a diblock copolymer including layers 201 and 202 within which the molecules of the diblock copolymer are oriented such that the hydrophobic “B” sections of the AB diblock copolymer are oriented towards each other and disposed within the membrane, while the hydrophilic “A” sections form the outer surfaces of the membrane. As such, membrane 301 may be considered to be a bilayer. Suitable methods of forming suspended membranes are known in the art, such as “painting”, e.g., brush painting (manual), mechanical painting (e.g., using stirring bar), and bubble painting (e.g., using flow through the device). Once formed, membrane 301 may be considered to be substantially “stable.” For example, membrane 301 may be sufficiently strong that a nanopore may not be inserted into the membrane without use of a chaotropic solvent to temporarily destabilize the membrane, e.g., in a manner as will now be described.

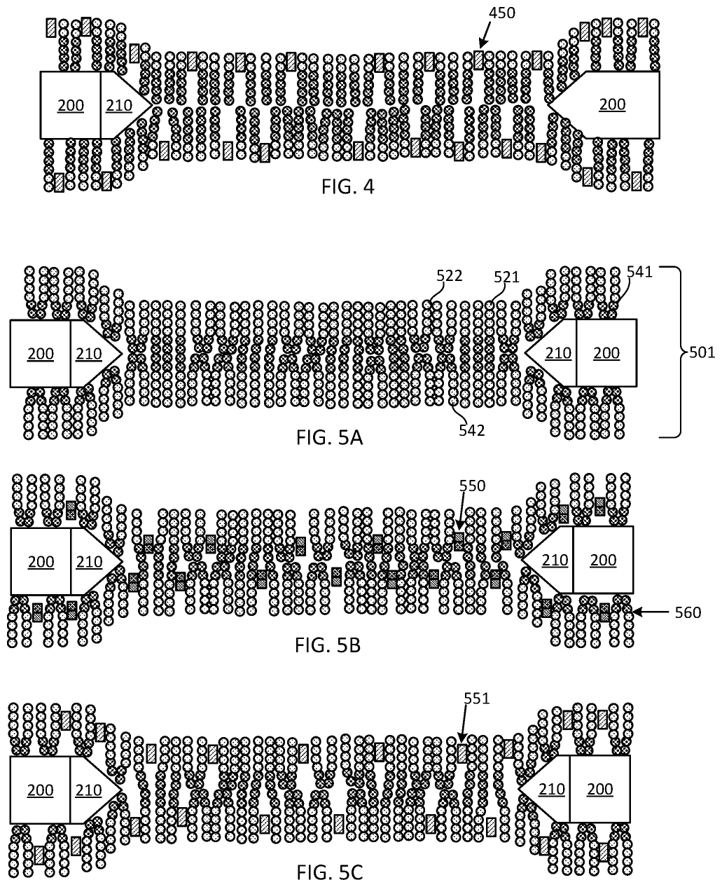

Turning now to FIG. 3B, a chaotropic solvent 350 is introduced to membrane 301, for example by adding the chaotropic solvent to the aqueous fluid(s) on the first side of the membrane, the second side of the membrane, or both the first and second side of the membrane (the latter being shown in FIG. 3B). Chaotropic solvent 350 is miscible with the fluid(s), e.g., is water-soluble. In the nonlimiting example illustrated in FIG. 3B, chaotropic solvent 350 includes an amphiphilic solvent, the molecules of which include a hydrophobic portion 351 and a hydrophilic portion 352. Alternatively, in a manner such as will be described with reference to FIG. 4, chaotropic solvent 350 may include a highly polar solvent. In still other examples, chaotropic solvent 350 may not necessarily be an amphiphilic solvent or a highly polar solvent, but may be used at a sufficiently high concentration within the aqueous fluid(s) to destabilize membrane 301. Nonlimiting examples of such chaotropic solvents are provided above in Table 1, and example concentrations of such solvents are provided as well.

In a manner such as illustrated in FIG. 3C, the chaotropic solvent 350 destabilizes membrane 301. For example, chaotropic solvent 350 intercalates between the polymer chains of polymeric membrane 301 in such a manner as to destabilize the membrane. Such intercalation may weaken intermolecular interactions within the membrane and/or may disrupt the arrangement of polymer chains within the membrane, or otherwise may significantly increase the free energy of the membrane. This causes the membrane to swell and loosen up, lowering its stability. In some examples, chaotropic solvent 350 may be soluble in the fluid(s) surrounding the membrane 301 and may be soluble in the block copolymer itself Illustratively, chaotropic solvents with an octanol-water partitioning coefficient value close to 0 (e.g., between about 2 and −2, or between about 1.5 and about −1.5, or between about 1 and about −1) may be used, as they may be expected to diffuse in and out of the block copolymer membrane relatively efficiently when adding and removing fluids in which the chaotropic solvent is mixed. Some examples of suitable amphiphilic solvents include isopropyl alcohol, n-butanol, ethanol, methanol, 1-propanol tetrahydrofuran, acetaldehyde, acetic acid, acetone, acetonitrile, ethylamine, and propanoic acid. As illustrated in FIG. 3C, chaotropic solvent 350 (e.g., amphiphilic solvent 350) intercalates between the polymer chains of membrane 301 to a region that is approximately at the interface 360 between hydrophilic and hydrophobic portions of those chains. In some examples, the membrane initially may be prepared in the destabilized state, e.g., as illustrated in FIG. 3C, and thus the operations described with reference to FIGS. 3A and 3B may be omitted or suitably modified such that the stable membrane need not be formed prior to destabilization. For example, the amphiphilic solvent (or other chaotropic solvent such as described elsewhere herein) may be dissolved in the liquids used to paint the membrane, and may intercalate between the polymer chains during initial formation of the membrane, thus destabilizing the membrane as that membrane is formed. In other examples in which chaotropic solvent 350 may not necessarily be an amphiphilic solvent or a highly polar solvent, an aqueous solution which includes the chaotropic solvent may be applied to a membrane which was formed at an earlier time.

Turning now to FIG. 3D, while the block copolymer membrane 301 is in the destabilized state, nanopore 110 may be inserted into the membrane more easily than if membrane 301 were in the stable state. For example, there may be weaker intermolecular interactions holding the individual molecules of the block copolymer membrane together in the destabilized state, allowing the polymer chains to more easily be moved apart to accommodate the nanopore being inserted. Nonlimiting examples of techniques for inserting nanopore 110 into the destabilized membrane include electroporation, pipette pump cycle, and detergent assisted nanopore insertion. Tools for forming membranes using synthetic polymers and inserting nanopores in the membranes are commercially available, such as the Orbit 16 TC platform available from Nanion Technologies Inc. (California, USA).

After nanopore insertion, membrane 301 may be stabilized by removing the chaotropic solvent from the system. For example, the chaotropic solvent may diffuse out of the membrane. Or, for example, the chaotropic solvent may be removed using a buffer wash. As discussed above, amphiphilic solvents with an octanol-water partitioning coefficient close to 0 (e.g., between about −2 and about 2, or between about −1.5 and about 1.5, or between about −1 and about 1) may be relatively easily removed from the membrane because they can diffuse in and out of the membrane relatively efficiently. The buffer wash may cause the chaotropic solvent 350 to diffuse out of the membrane and into the buffer. The buffer wash may include multiple washes, e.g., so as to remove enough of the chaotropic solvent so that membrane stability returns. Removal of the chaotropic solvent (whether by washing or by diffusion without the need for washing) stabilizes the block copolymer membrane by allowing the intermolecular interactions within the membrane to resume. This allows the membrane to regain its stable properties yet have a nanopore inserted therein for use in a device, e.g., such as described with reference to FIGS. 1 and 2A-2B.

It will be appreciated that any suitable chaotropic solvent may be used to destabilize membrane 301, and that an amphiphilic solvent, or a solvent which is used at a sufficiently high concentration with an already-formed membrane, are nonlimiting examples. FIG. 4 illustrates the operation described with reference to FIG. 3C, using a different type of chaotropic solvent. In the nonlimiting example illustrated in FIG. 4, highly polar chaotropic solvent 450 is used to destabilize membrane 301 by intercalating between the hydrophilic blocks of the polymer chains, thus weakening intermolecular interactions, disrupting the arrangement of polymer chains within the membrane, or otherwise substantially increasing the free energy of the membrane. Some examples of highly polar chaotropic solvents include dimethylsulfoxide, acetonitrile, urea, acetonitrile, formamide, dimethylformamide, methyl isocyanide, N-methyl-2-pyrrolidone, and triethylene glycol. Because the hydrophilic regions are on the outside of the membrane, highly polar chaotropic solvents may be able to diffuse in and out of the hydrophilic region of the block copolymer membrane efficiently when adding and removing solutions containing the highly polar chaotropic solvent in a manner similar to that described with reference to FIGS. 3A-3D.

Although FIGS. 3A-3D illustrate operations for inserting a nanopore into a membrane including a diblock copolymer, it will be appreciated that such operations similarly may be used with membranes that include other types of polymers. For example, FIGS. 5A-5C illustrate operations for inserting a nanopore into another example polymeric membrane using different types of chaotropic solvents. FIG. 5A illustrates membrane 501 including molecules of an ABA triblock copolymer including hydrophobic “B” sections 541 coupled to and between hydrophilic “A” sections 542. Each individual ABA molecule may be in one of two arrangements. For example, ABA molecules 521 may extend through the layer in a linear fashion, with an “A” section on each side of the membrane and the “B” section in the middle of the membrane. Or, for example, ABA molecules 522 may extend to the middle of the membrane and then fold back on themselves, so that both “A” sections are on the same side of the membrane and the “B” section is in the middle of the membrane. Accordingly, the example shown in FIG. 5A may be considered to be partially a single layer and partially a bilayer. Accordingly, the polymer membrane may be considered to include at least one layer comprising a plurality of molecules of the triblock copolymer, the first hydrophilic blocks and the second hydrophilic blocks of the second plurality of molecules forming first and second outer surfaces of the polymer membrane. Different types of chaotropic solvents may be used to destabilize membrane 501. For example, as illustrated in FIG. 5B, the chaotropic solvent may include an amphiphilic solvent 550 that intercalates to interface 560 between the A and B blocks. Or, for example, as illustrated in FIG. 5C, the chaotropic solvent may include a highly polar solvent 551 that intercalates between the A blocks. Or, for example, the chaotropic solvent may include a solvent that is not necessarily an amphiphilic solvent or a chaotropic solvent, and that intercalates to any suitable location between the A blocks, B blocks, and/or an interface between the A and B blocks.

In another example, FIGS. 6A-6C illustrate operations for inserting a nanopore into another example polymeric membrane using different types of chaotropic solvents. FIG. 6A illustrates membrane 601 including molecules of a BAB triblock copolymer including hydrophilic “A” sections 642 coupled to and between hydrophobic “B” sections 641. In this example, membrane 601 may have a bilayer architecture with the “B” sections 641 oriented towards each other. The hydrophobic ends of the BAB molecules generally may be located approximately in the middle of membrane 601, the molecules then extend towards either outer surface of the membranes, and then fold back on themselves. As such, both “B” sections are located in the middle of the membrane and the “A” section is on one side or the other of the membrane, and the membrane may be considered to be a bilayer. Different types of chaotropic solvents may be used to destabilize membrane 601. For example, as illustrated in FIG. 6B, the chaotropic solvent may include an amphiphilic solvent 650 that intercalates to interface 660 between the A and B blocks. Or, for example, as illustrated in FIG. 6C, the chaotropic solvent may include a highly polar solvent 651 that intercalates between the A blocks. Or, for example, the chaotropic solvent may include a solvent that is not necessarily an amphiphilic solvent or a chaotropic solvent, and that intercalates to any suitable location between the A blocks, B blocks, and/or an interface between the A and B blocks.

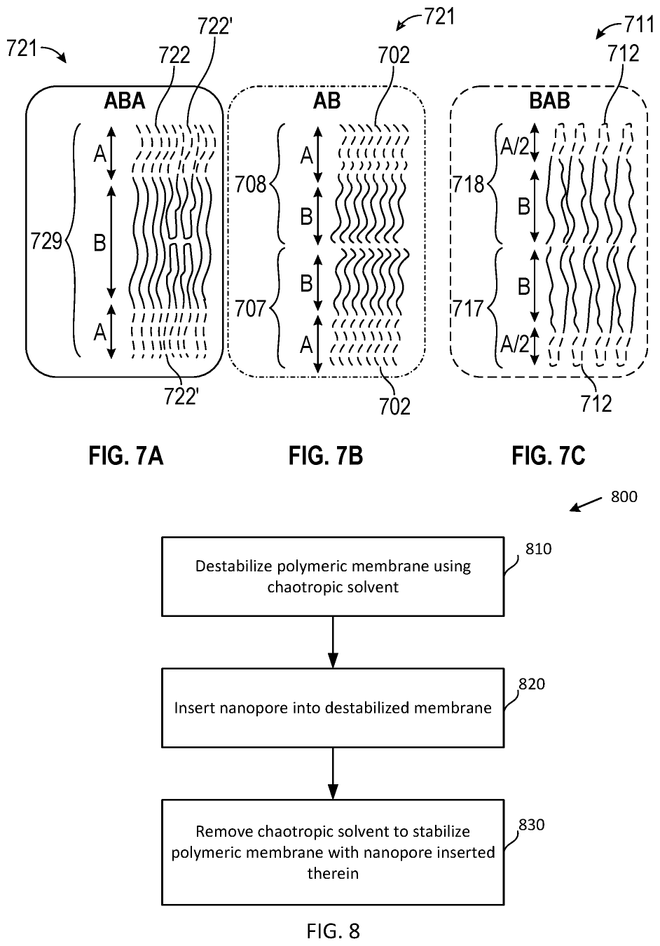

FIGS. 7A-7C schematically illustrate further details of membranes using block copolymers which may be included in the nanopore composition and device of FIG. 1 and used in respective operations described with reference to FIGS. 3A-6C. It will be appreciated that such membranes suitably may be adapted for use in any other composition or device, and are not limited to use with nanopores.

Referring now to FIG. 7B, membrane 701 uses a diblock “AB” copolymer. Membrane 701 includes first layer 707 which may contact fluid 120 and second layer 708 which may contact fluid 120′ in a manner similar to that described with reference to FIG. 1. First layer 707 includes a first plurality of molecules 702 of a diblock AB copolymer, and second layer 708 includes a second plurality of the molecules 702 of the diblock AB copolymer. As illustrated in FIG. 7B, each molecule 702 of the diblock copolymer includes a hydrophobic block, denoted “B” and being approximately of length “B,” coupled to a hydrophilic block, denoted “A” and being approximately of length “A”. The hydrophilic A blocks of the first plurality of molecules 702 (the molecules forming layer 707) form a first outer surface of the membrane 701, e.g., contact fluid 120. The hydrophilic A blocks of the second plurality of molecules 702 (the molecules forming layer 708) form a second outer surface of the membrane 702, e.g., contact fluid 120′. The respective ends of the hydrophobic B blocks of the first and second pluralities of molecules contact one another within the membrane 701 in a manner such as illustrated in FIG. 7B. As illustrated, substantially all of the molecules 702 within layer 707 may extend substantially linearly and in the same orientation as one another, and similarly substantially all of the molecules 702 within layer 708 may extend substantially linearly and in the same orientation as one another (which is opposite that of the orientation the molecules within layer 707). Accordingly, first and second layers 707, 708 each may have a thickness of approximately A+B, and membrane 701 may have a thickness of approximately 2A+2B. In some examples, length A is about 2 repeating units (RU) to about 100 RU, or about 1 repeating unit (RU) to about 50 RU, e.g., about 5 RU to about 40 RU, or about 10 RU to about 30 RU, or about 10 RU to about 20 RU, or about 20 RU to about 40 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. Optionally, barrier 701 described with reference to FIG. 7B may be suspended across an aperture in a manner such as described with reference to FIGS. 2A-2B and 3A-3D.

Referring now to FIG. 7C, membrane 711 uses a triblock “BAB” copolymer. Membrane 711 includes first layer 717 which may contact fluid 120 and second layer 718 which may contact fluid 120′ in a manner similar to that described with reference to FIG. 1. First layer 717 includes a first plurality of molecules 712 of a triblock copolymer, and second layer 718 includes a second plurality of the molecules 712 of the triblock copolymer. As illustrated in FIG. 7C, each molecule 712 of the triblock copolymer includes first and second hydrophobic blocks, each denoted “B” and being approximately of length “B,” and a hydrophilic block disposed between the first and second hydrophobic blocks, denoted “A” and being approximately of length “A”. The hydrophilic A blocks of the first plurality of molecules 712 (the molecules forming layer 717) form a first outer surface of the membrane 711, e.g., contact fluid 120. The hydrophilic A blocks of the second plurality of molecules 712 (the molecules forming layer 718) form a second outer surface of the membrane 711, e.g., contact fluid 120′. The respective ends of the hydrophobic B blocks of the first and second pluralities of molecules contact one another within the membrane 711 in a manner such as illustrated in FIG. 7C. As illustrated, substantially all of the molecules 712 within layer 717 may extend in the same orientation as one another, and may be folded at the A block so that the A block can contact the fluid while the B blocks are interior to the membrane 711. Similarly, substantially all of the molecules 712 within layer 718 may extend in the same orientation as one another (which is opposite that of the orientation the molecules within layer 717), and may be folded at their A blocks so that the A blocks contact the fluid while the B blocks are interior to the membrane 711. Accordingly, first and second layers 717, 718 each may have a thickness of approximately A/2+B, and membrane 711 may have a thickness of approximately A+2B. In some examples, length A is about 2 RU to about 100 RU, e.g., about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. Optionally, barrier 711 described with reference to FIG. 7C may be suspended across an aperture in a manner such as described with reference to FIGS. 6A-6C.

Referring now to FIG. 7A, membrane 721 uses a triblock “ABA” copolymer. Membrane 721 includes layer 729 which may contact both fluids 120 and 120′. Layer 729 includes a plurality of molecules 722 of a triblock ABA copolymer. As illustrated in FIG. 7A, each molecule 722 of the triblock copolymer includes first and second hydrophilic blocks, each denoted “A” and being approximately of length “A,” and a hydrophobic block disposed between the first and second hydrophilic blocks, denoted “B” and being approximately of length “B”. The hydrophilic A blocks at first ends of molecules 722 (the molecules forming layer 729) form a first outer surface of the membrane 721, e.g., contact fluid 120. The hydrophilic A blocks at second ends of molecules 722 form a second outer surface of the membrane 721, e.g., contact fluid 120′. The hydrophobic B blocks of the molecules 722 are within the membrane 711 in a manner such as illustrated in FIG. 7C. As illustrated, the majority of molecules 722 within layer 729 may extend substantially linearly and in the same orientation as one another. Optionally, as illustrated in FIG. 7A, some of the molecules 722′ may be folded at their B blocks, such that both of the hydrophilic A blocks of such molecules may contact the same fluid as one another. Accordingly, the example shown in FIG. 7A may be considered to be partially a single layer, and partially a bilayer. In other examples (not specifically illustrated), layer 729 may be entirely a single-layer or may be entirely a bilayer, e.g., as also described with reference to FIG. 1. Regardless of whether the membrane includes molecules 722 which extend substantially linearly and/or molecules 222′ which are folded, as illustrated in FIG. 7A, layer 729 may have a thickness of approximately 2A+B, and the polymer membrane may be considered to include at least one layer comprising a plurality of molecules of the triblock copolymer, the first hydrophilic blocks and the second hydrophilic blocks of the second plurality of molecules forming first and second outer surfaces of the polymer membrane. In some examples, length A is about 1 RU to about 100 RU, e.g., about 2 RU to about 100 RU, or about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. It will be appreciated that any end groups that are coupled to the hydrophilic or hydrophobic blocks contribute to the overall thickness of the barrier. Optionally, barrier 721 described with reference to FIG. 2A may be suspended across an aperture in a manner such as described with reference to FIGS. 5A-5C.

It will be appreciated that the layers of the various membranes provided herein may be configured so as to have any suitable dimensions. Illustratively, to form membranes of similar dimension as one another:

A-B-A triblock copolymer (FIG. 7A) may have 2 hydrophilic blocks, each of length A (each A block is of M_{w}=x) and 1 hydrophobic block of length B (M_{w}=y); when self-assembled, those A-B-A triblock copolymers would form membranes with a top hydrophilic layer of length A, a core hydrophobic layer of length B, and a bottom hydrophilic layer of length A.

A-B diblock copolymer (FIG. 7B) may have 1 hydrophilic block of length A (M_{w}=x), and 1 hydrophobic block of length B (M_{w}=y/2); when self-assembled, those A-B diblock copolymers would form membranes with a top hydrophilic layer of length A, a core hydrophobic layer of length 2B, and a bottom hydrophilic layer of length A.

B-A-B triblock copolymer (FIG. 7C) may have 1 hydrophilic block of length A (M_{w}=x), and 2 hydrophobic blocks, each of length of B (each B block is of M_{w}=y/2); when self-assembled, those B-A-B triblock copolymers would form membranes with a top hydrophilic layer of length A/2, a core hydrophobic layer of length 2B, and a bottom hydrophilic layer of length A/2.

Additionally, or alternatively, the polymer packing into the layer(s) of the membrane may affect the hydrophilic ratio for each of the membranes, where hydrophilic ratio may be defined as the ratio between molecular mass of the hydrophilic block and the total molecular weight (MW or M_{w}) of the block copolymer (BCP) (hydrophilic ratio=Mw hydrophilic block/M_{w} BCP). For example:

A-B-A triblock copolymer (FIG. 7A), hydrophilic ratio=2x/(2x+y);

A-B diblock copolymer (FIG. 7B), hydrophilic ratio=x/(x+y/2); and

B-A-B triblock copolymer (FIG. 7C), hydrophilic ratio=x/(x+y).

The present diblock and triblock copolymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

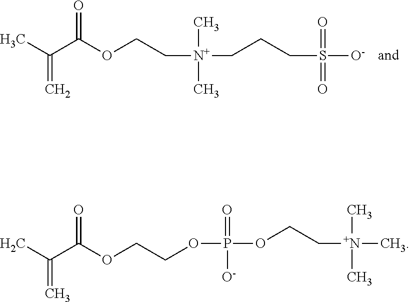 [SMILES: C=C(C)C(=O)OCC[N+](C)(C)CCCS(=O)(=O)[O-] ; "and" ; C=C(C)C(=O)OCCOP(=O)([O-])OCC[N+](C)(C)C]

Nonlimiting examples of hydrophilic polypeptides include:

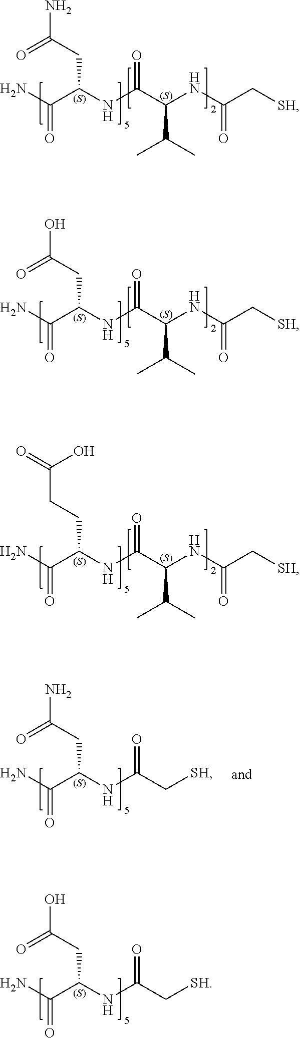

A nonlimiting example of a charged polyacrylamide is

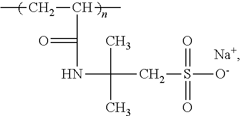

where n is between about 2 and about 100. Nonlimiting examples of nitrogen containing units include:

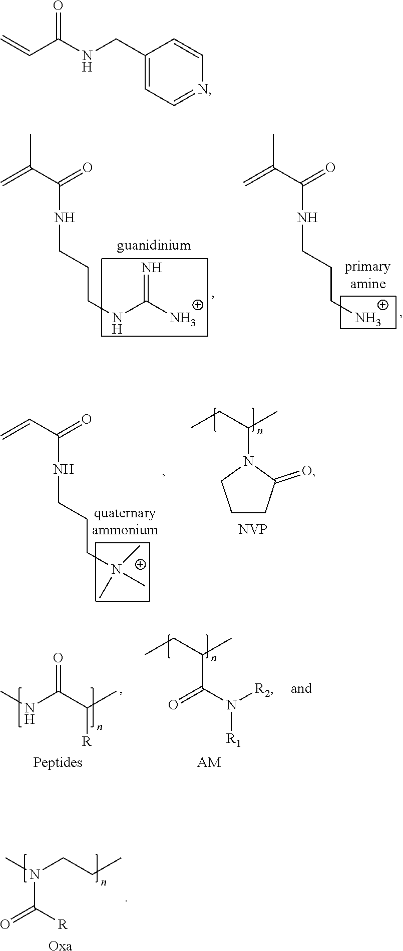

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

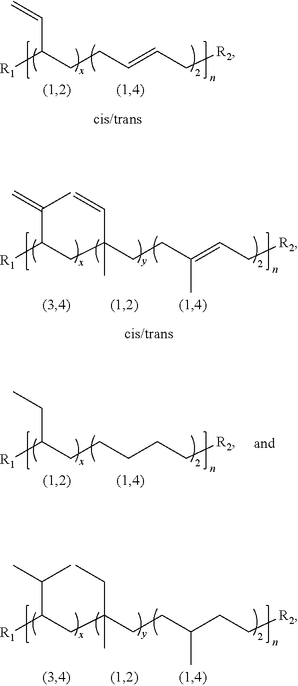

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting example of fluorinated polyethylene is

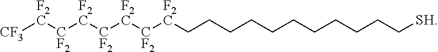 [SMILES: FC(F)(F)C(F)(F)C(F)(F)C(F)(F)C(F)(F)C(F)(F)C(F)(F)C(F)(F)CCCCCCCCCCCS]

Nonlimiting examples of hydrophobic polypeptides include (0<x<1):

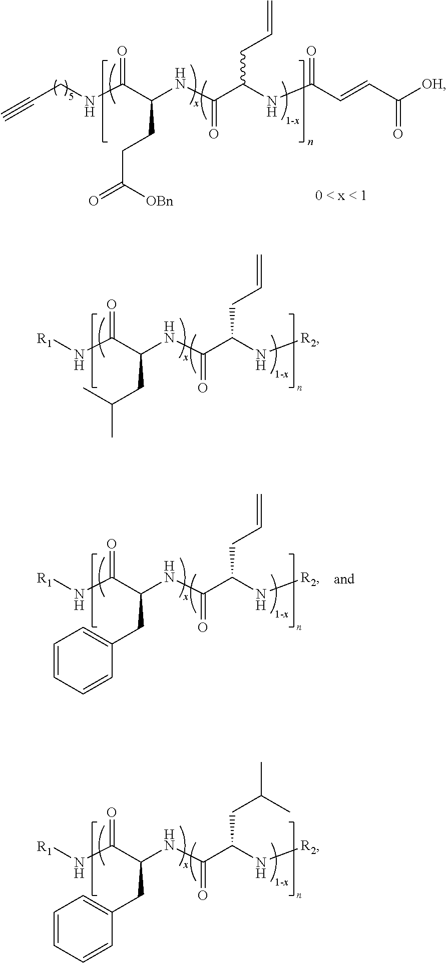

where n is between about 2 and about 100.

In one nonlimiting example, an AB diblock copolymer includes PDMS-b-PEO, where “-b-” denotes that the polymer is a block copolymer. In another nonlimiting example, an AB diblock copolymer includes PBd-b-PEO. In another nonlimiting example, an AB diblock copolymer includes PIB-b-PEO. In another nonlimiting example, a BAB triblock copolymer includes PDMS-b-PEO-b-PDMS. In another nonlimiting example, a BAB triblock copolymer includes PBd-b-PEO-b-PBd. In another nonlimiting example, a BAB triblock copolymer includes PIB-b-PEO-b-PIB. In another nonlimiting example, an ABA triblock copolymer includes PEO-b-PBd-b-PEO. In another nonlimiting example, an ABA triblock copolymer includes PEO-b-PDMS-b-PEO. In another nonlimiting example, an ABA triblock copolymer includes PEO-b-PIB-b-PEO. It will be appreciated that any suitable hydrophilic block(s) may be used with any suitable hydrophobic block(s). Additionally, in examples including two hydrophilic blocks, those blocks may be but need not necessarily include the same polymers as one another. Similarly, in examples including two hydrophobic blocks, those blocks may be but need not necessarily include the same polymers as one another.

The respective molecular weights, glass transition temperatures, and chemical structures of the hydrophobic and hydrophilic blocks suitably may be selected so as to provide the membrane with appropriate stability for use and ability to insert a nanopore, e.g., while the membrane is destabilized using a chaotropic solvent. For example, the respective molecular weights of the hydrophobic and hydrophilic blocks may affect how thick each of the blocks (and thus layers of the membrane) are, and may influence stability as well as capacity to insert the nanopore while the membrane is destabilized using a chaotropic solvent, e.g., through electroporation, pipette pump cycle, or detergent assisted nanopore insertion. Additionally, or alternatively, the ratio of molecular weights of the hydrophilic and hydrophobic blocks may affect self-assembly of those blocks into the layers of the membrane. Additionally, or alternatively, the respective glass transition temperatures (T_{g}) of the hydrophobic and hydrophilic blocks may affect the lateral fluidity of the layers of the membrane; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers in the stable state and in the destabilized state.

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around ° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

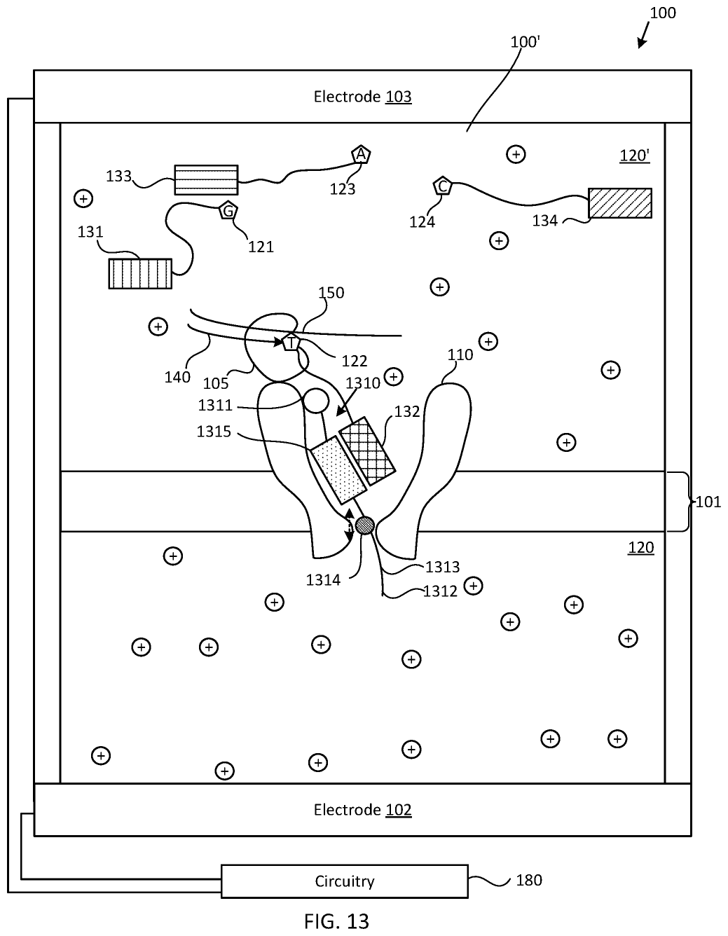

Hydrophobic B blocks with a low T_{g} may be used to help maintain membrane flexibility under conditions suitable for performing nanopore sequencing, e.g., in a manner such as described with reference to FIGS. 13-17. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIGS. 13-17).

In some examples of the AB copolymer shown below including PBd as the B block and PEO as the A block, R is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen; m=about 2 to about 100; and n=about 2 to about 100.

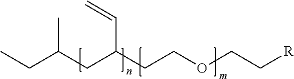

In some nonlimiting examples, R=OH; n=about 8 to about 50; and m=about 1 to about 20. In some nonlimiting examples, R=OH; n=about 10 to about 15; and m=about 5 to about 15.

In some examples of the ABA copolymer shown below including one or more PM blocks as the B block and PEO as the A block, R₁ and R₂ are independently moieties selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen; V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about 20 carbons, or another aliphatic group; m=about 2 to about 100; and n=about 2 to about 100. V may optionally be flanked by functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. When V is absent, only one PIB block is present and n=about 2 to about 100. L₁ and L₂ are independently linkers, which may include at least one moiety selected from the group consisting of an amide, a thioether (sulfide), a succinic group, a maleic group, an alkyl group (such as a methylene), an ether, and a product of a “click” reaction.

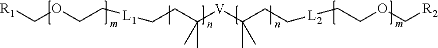

In some nonlimiting examples of the above structure, n=about 2 to about 50, and m=about 1 to about 50, R₁=R₂=COOH, V=tert-butylbenzene, and L₁=L₂=ethyl sulfide. In other nonlimiting examples, n=about 5 to about 20, m=about 2 to about 15, R₁=R₂=COOH, V=tert-butylbenzene, and L₁=L₂=ethyl sulfide. In other nonlimiting examples, n=about 13 to about 19, m=about 2 to about 5, R₁=R₂=COOH, V=tert-butylbenzene, and L₁=L₂=ethyl sulfide. In other nonlimiting examples, n=about 7 to about 13, m=about 7 to about 13, R₁=R₂=COOH, V=tert-butylbenzene, and L₁=L₂=ethyl sulfide. In particular, in one nonlimiting example (the structure of which is shown below), n=16, m=3, R₁=R₂=COOH, V=tert-butylbenzene, and L₁=L₂=ethyl sulfide. In another nonlimiting example (the structure of which is shown below, n =10, m=10, and R₁=R₂=COOH, V=tert-butylbenzene, and L₁=L₂=ethyl sulfide. In another nonlimiting example (the structure of which is shown below), n=16, m=8, R₁=R₂=methyl, V =tert-butylbenzene, and L₁=L₂=ethyl sulfide.

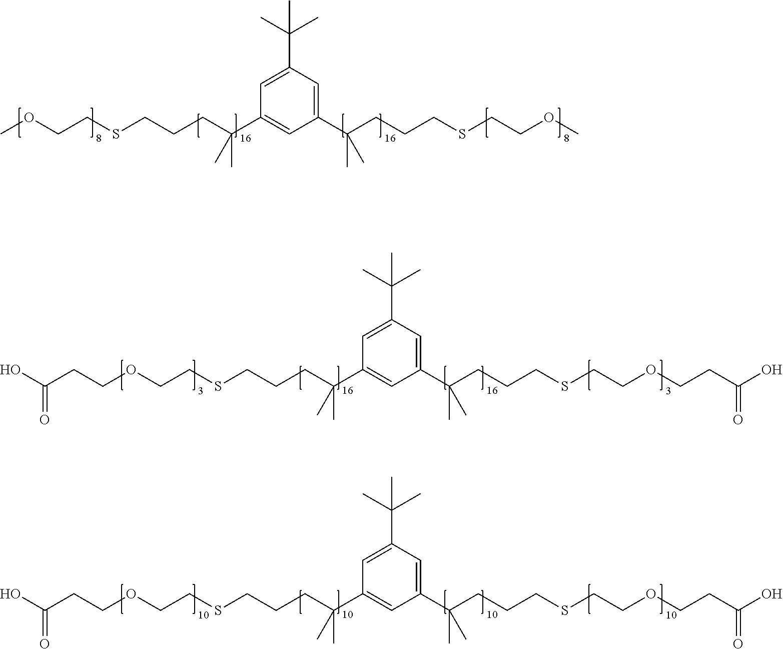

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another nonlimiting example, an ABA triblock copolymer includes

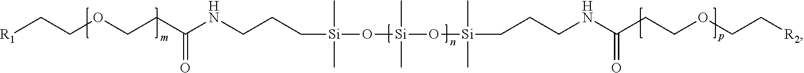

where m=about 2 to about 100, n=about 2 to about 100, p=about 2 to about 100, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. In some nonlimiting examples, m=about 2 to about 30, n=about 25 to about 45, p=about 2 to about 30, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. In some nonlimiting examples, m=about 2 to about n=about 30 to about 40, p=about 2 to about 15, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. In some nonlimiting examples, m=about 7 to about 11, n=about 35 to about 40, p=about 7 to about 11, R₁ and R₂ are independently functional groups selected from the group consisting of a a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. In some nonlimiting examples, m=about 2 to about 5, n=about to about 37, p=about 2 to about 5, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen.

In particular, as shown below, in one nonlimiting example, m=3, n=34, p=3, and R₁=R₂=COOH. In another nonlimiting example shown below, m=9, n=37, p=9, and R₁=R₂=COOH.

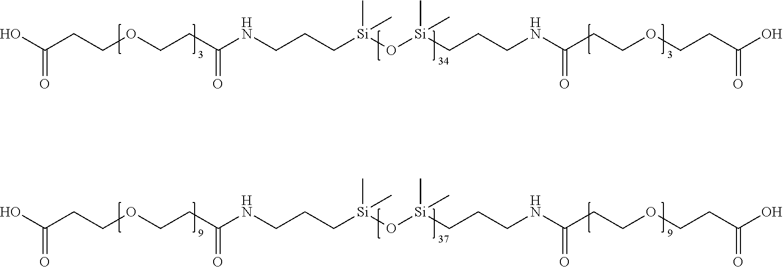

In some examples of the AB copolymer shown below including a PIB block as the B block and PEO as the A block, R is a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen; m=about 2 to about 100; n=about 2 to about 100; and L is a linker selected from the group consisting of an amide, a thioether (sulfide), a succinic group, a maleic group, an alkyl group (such as a methylene), an ether, or a product of a click reaction.

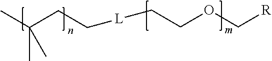

In particular, as shown below, in one nonlimiting example, n=13, m=8, R is methyl, and L is ethyl sulfide. In another nonlimiting example shown below, n=13, m=3, R is a carboxyl group, and L is ethyl sulfide. In another nonlimiting example shown below, n=30, m=8, R is methyl, and L is ethyl sulfide. In another nonlimiting example shown below, n=30, m=3, R is a carboxyl group, and L is ethyl sulfide.

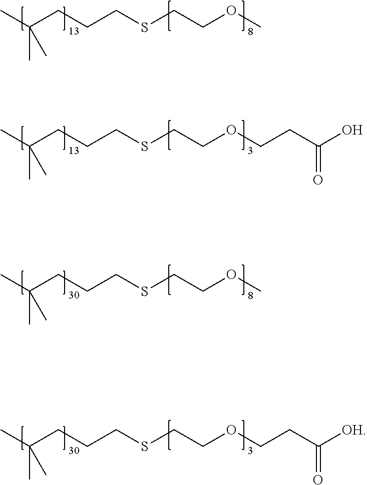

Accordingly, it will be appreciated that a wide variety of polymeric membranes may be destabilized using a suitable chaotropic solvent so as to facilitate nanopore insertion into such membranes. FIG. 8 illustrates an example flow of operations in a method 800 for inserting a nanopore into a polymer membrane. Method 800 may include destabilizing the polymer membrane using a chaotropic solvent (operation 810). For example, polymer membrane 101, 301, 501, 601, 701, 711, or 721 may be in a substantially stable state, and may be contacted by a fluid within which a chaotropic solvent is dissolved. Or, for example, the polymer membrane may be formed in a destabilized state. In either case, the chaotropic solvent may intercalate between chains of the polymer membrane and thus destabilize the membrane. For example, the chaotropic solvent may include an amphiphilic solvent that intercalates to interfaces between the hydrophilic blocks and the hydrophobic blocks, e.g., in a manner such as described with reference to FIGS. 3C, 5B, or 6B. Or, for example, the chaotropic solvent may include a highly polar solvent that intercalates to between the hydrophilic blocks, e.g., in a manner such as described with reference to FIGS. 4, 5C, or 6C. Or, for example, the chaotropic solvent may include an amphiphilic solvent that intercalates to interfaces between the hydrophilic blocks and the hydrophobic blocks, e.g., in a manner such as described with reference to FIGS. 3C, 5B, or 6B, and a highly polar solvent that intercalates to interfaces between the hydrophilic block, e.g., in a manner such as described with reference to FIGS. 4, 5C, or 6C. In some examples, a combination of such amphiphilic solvent(s) and highly polar solvent(s). For example, the amphiphilic solvent may be used to destabilize the hydrophobic blocks and the highly polar solvent may be used to destabilize the hydrophilic blocks.

Method 800 illustrated in FIG. 8 also may include inserting the nanopore into the destabilized membrane (operation 820). For example, the nanopore may be inserted into any of the present destabilized membranes in a manner such as described with reference to FIG. 3D. any suitable nanopore insertion technique may be used to insert the nanopore while the membrane is destabilized by the chaotropic solvent. The destabilization of the membrane may facilitate insertion of the nanopore as compared to insertion into the same membrane in the stable state. Method 800 illustrated in FIG. 8 also may include removing the chaotropic solvent to stabilize the polymer membrane with the nanopore inserted therein (operation 830). For example, any suitable number of buffer washes may be used to dissolve the chaotropic solvent out of the membrane, thus stabilizing the membrane. Alternatively, the chaotropic solvent may diffuse out of the membrane without the need for such washes.

It will further be appreciated that the present barriers may be used in any suitable device or application. For example, FIG. 13 schematically illustrates a cross-sectional view of an example use of the composition and device of FIG. 1. Device 100 illustrated in FIG. 13 may be configured may include fluidic well 100′, barrier 101 which may have a configuration such as described elsewhere herein, first and second fluids 120, 120′, and nanopore 110 in a manner such as described with reference to FIG. 1. In the nonlimiting example illustrated in FIG. 13, second fluid 120′ optionally may include a plurality of each of nucleotides 121, 122, 123, 124, e.g., G, T, A, and C, respectively. Each of the nucleotides 121, 122, 123, 124 in second fluid 120′ optionally may be coupled to a respective label 131, 132, 133, 134 coupled to the nucleotide via an elongated body (elongated body not specifically labeled). Optionally, device 100 further may include polymerase 105. As illustrated in FIG. 13, polymerase 105 may be within the second composition of second fluid 120′. Alternatively, polymerase 105 may be coupled to nanopore 110 or to barrier 101, e.g., via a suitable elongated body (not specifically illustrated). Device 100 optionally further may include first and second polynucleotides 140, 150 in a manner such as illustrated in FIG. 13. Polymerase 105 may be for sequentially adding nucleotides of the plurality to the first polynucleotide 140 using a sequence of the second polynucleotide 150. For example, at the particular time illustrated in FIG. 13, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150.

Circuitry 180 illustrated in FIG. 13 may be configured to detect changes in an electrical characteristic of the aperture responsive to the polymerase sequentially adding nucleotides of the plurality to the first polynucleotide 140 using a sequence of the second polynucleotide 150. In the nonlimiting example illustrated in FIG. 13, nanopore 110 may be coupled to permanent tether 1310 which may include head region 1311, tail region 1312, elongated body 1313, reporter region 1314 (e.g., an abasic nucleotide), and moiety 1315. Head region 1311 of tether 1310 is coupled to nanopore 110 via any suitable chemical bond, protein-protein interaction, or any other suitable attachment that is normally irreversible. Head region 1311 can be attached to any suitable portion of nanopore 110 that places reporter region 1314 within aperture 1313 and places moiety 1315 sufficiently close to polymerase 105 so as to interact with respective labels 131, 132, 133, 134 of nucleotides 121, 122, 123, 124 that are acted upon by polymerase 105. Moiety 1315 respectively may interact with labels 131, 132, 133, 134 in such a manner as to move reporter region 1314 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

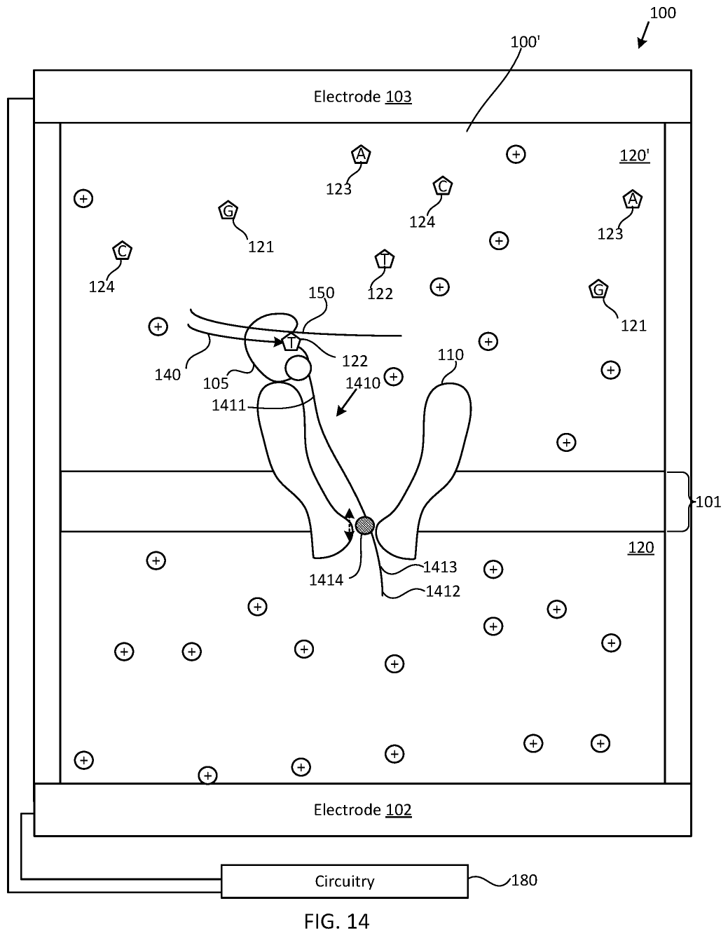

FIG. 14 schematically illustrates a cross-sectional view of another example use of the composition and device of FIG. 1. As illustrated in FIG. 14, device 100 may include fluidic well 100′, barrier 101 which may have a configuration such as described elsewhere herein, first and second fluids 120, 120′, nanopore 110, and first and second polynucleotides 140, 150, all of which may be configured similarly as described with reference to FIG. 13. In the nonlimiting example illustrated in FIG. 14, nucleotides 121, 122, 123, 124 need not necessarily be coupled to respective labels. Polymerase 105 may be coupled to nanopore 110 and may be coupled to permanent tether 1410 which may include head region 1411, tail region 1412, elongated body 513, and reporter region 1414 (e.g., an abasic nucleotide). Head region 1411 of tether 1410 is coupled to polymerase 105 via any suitable chemical bond, protein-protein interaction, or any other suitable attachment that is normally irreversible. Head region 1411 can be attached to any suitable portion of polymerase 105 that places reporter region 1414 within aperture 113. As polymerase 105 interacts with nucleotides 121, 122, 123, 124, such interactions may cause polymerase 105 to undergo conformational changes. Such conformational changes may move reporter region 1414 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

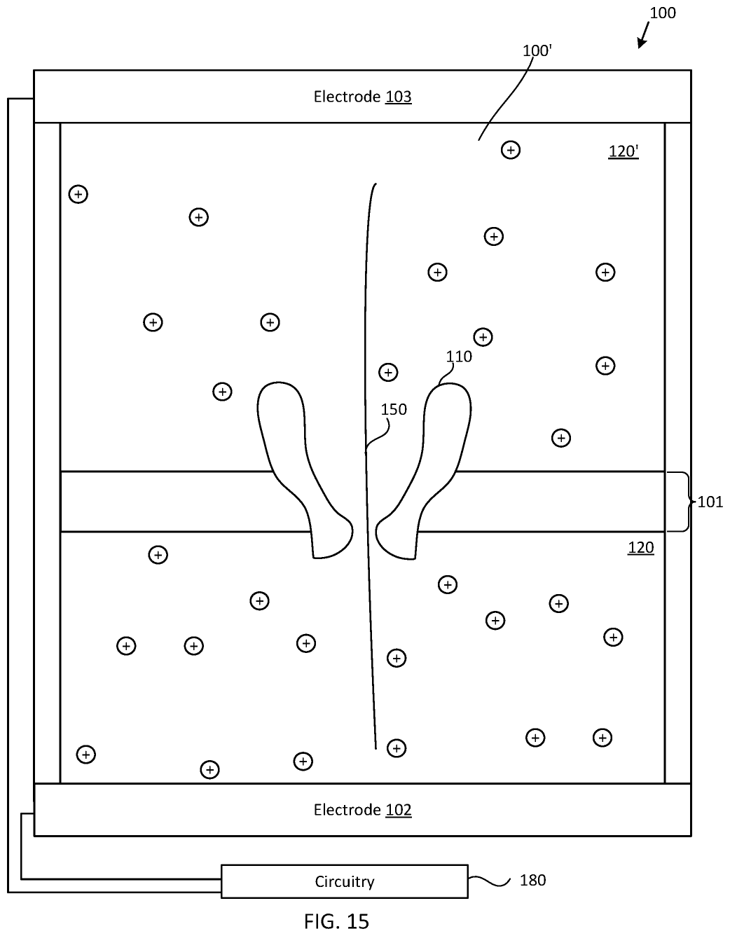

FIG. 15 schematically illustrates a cross-sectional view of another example use of the composition and device of FIG. 1. As illustrated in FIG. 15, device 100 may include fluidic well 100′, barrier 101 which may have a configuration such as described elsewhere herein, first and second fluids 120, 120′, and nanopore 110 all of which may be configured similarly as described with reference to FIG. 13. In the nonlimiting example illustrated in FIG. 15, polynucleotide 150 is translocated through nanopore 110 under an applied force, e.g., a bias voltage that circuitry 180 applies between electrode 102 and electrode 103. As bases in polynucleotide 150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

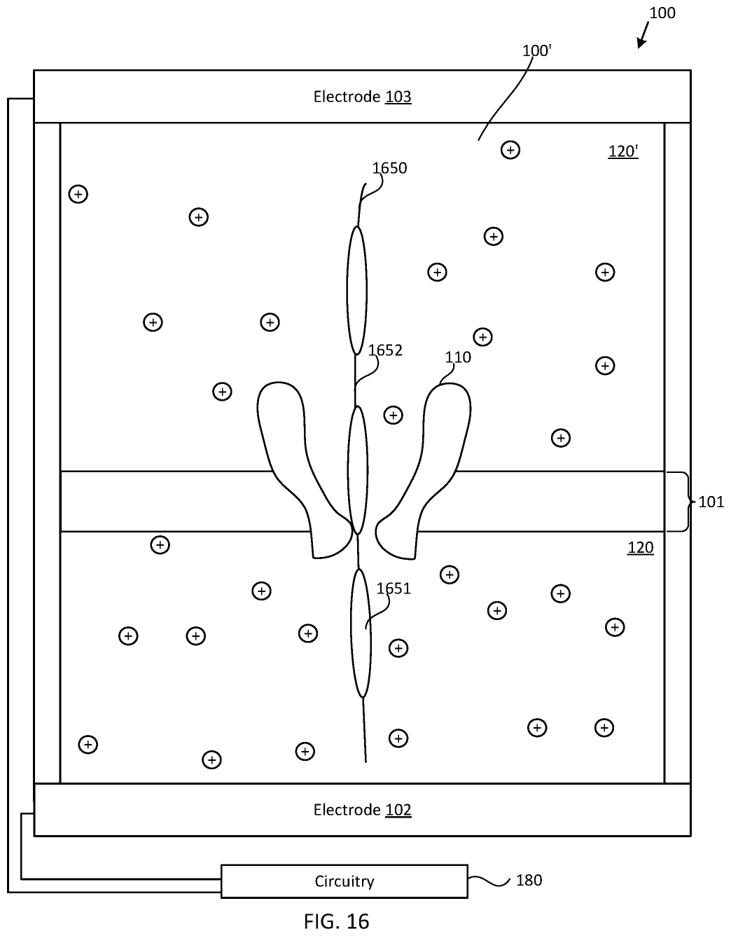

FIG. 16 schematically illustrates a cross-sectional view of another example use of the composition and device of FIG. 1. As illustrated in FIG. 16, device 100 may include fluidic well 100′, barrier 101 which may have a configuration such as described elsewhere herein, first and second fluids 120, 120′, and nanopore 110 all of which may be configured similarly as described with reference to FIG. 13. In the nonlimiting example illustrated in FIG. 16, surrogate polymer 1650 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 16, surrogate polymer 1650 includes labels 1651 coupled to one another via linkers 1652. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,457,979, 10,676,782, 10,745,685, 10,774,105, and U.S. Pat. No. 10,851,405.

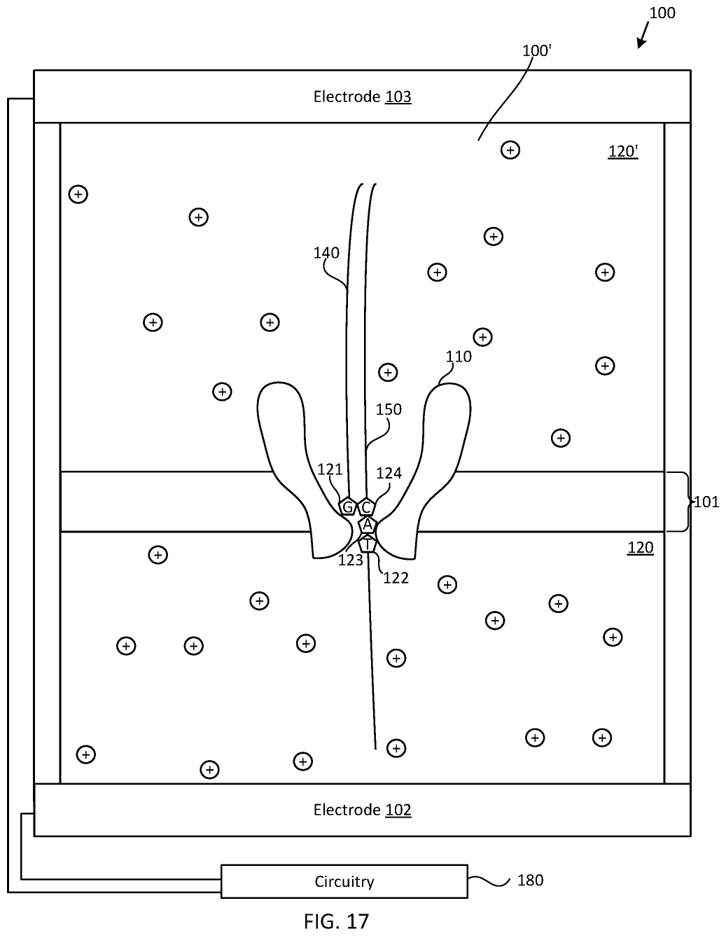

FIG. 17 schematically illustrates a cross-sectional view of another example use of the composition and device of FIG. 1. As illustrated in FIG. 17, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 all of which may be configured similarly as described with reference to FIG. 4. In the nonlimiting example illustrated in FIG. 17, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

### WORKING EXAMPLES

The following examples are intended to be purely illustrative, and not limiting of the present invention.

The performance of the ABA triblock copolymer poly(ethylene oxide)-b-poly(dimethyl siloxane)-b-poly(ethylene oxide) (PEO-b-PDMS-b-PEO) was assessed in terms of membrane stability both in the presence of and in the absence of a chaotropic solvent. Isopropyl alcohol and n-butanol were used as chaotropic solvents. Membranes were generated using an automated patch clamp device using Ag/AgCl electrodes.

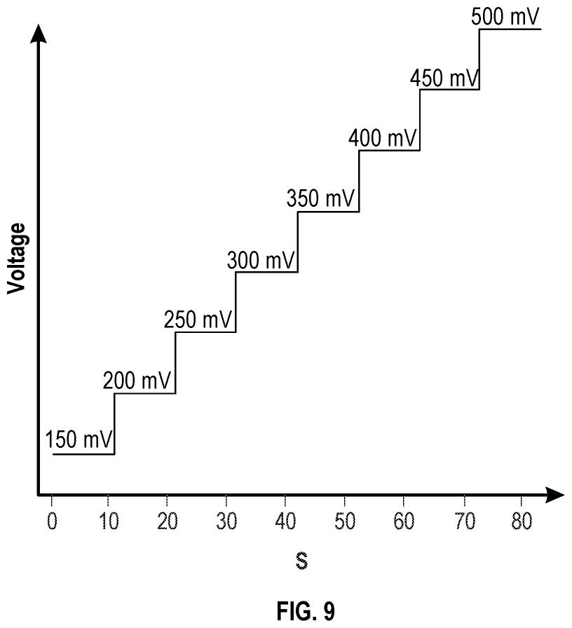

FIG. 9 illustrates the voltage breakdown waveform used to assess polymeric membrane stability. Membrane stability was quantified as the percentage of membranes remaining at the end of each step of the voltage ramp illustrated. The voltage ramp was stepped in 50 mV steps from 150 mV to 500 mV, as shown in FIG. 9. Each step lasted for 10 seconds. Nanopore insertion was represented as the number of successful single nanopore insertions during each individual experiment with a maximum of 16 nanopores per experiment. The membranes were generated under standard buffer conditions (1M KCl, 50 mM HEPES, pH=7.4).

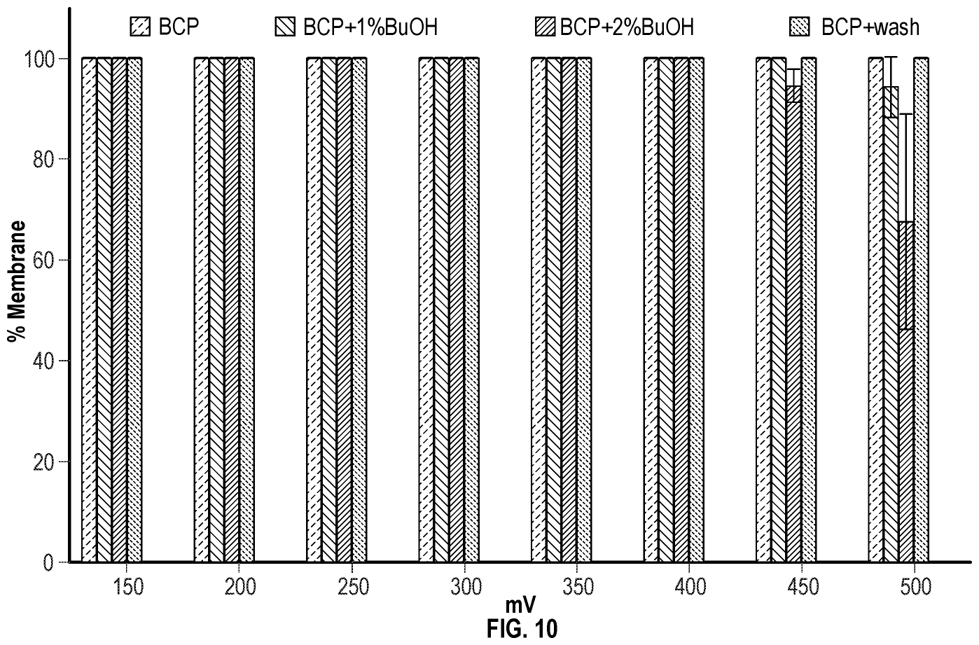

FIG. 10 is a plot showing the measured membrane stability of the example suspended copolymeric membrane generated in buffer solutions with different n-butanol content. As can be seen from FIG. 10, suspended PEO-PDMS-PEO membranes generated under standard buffer conditions (1M KCl, 50 mM 2-[4-(2-hydroxyethyl)piperazin-1-yl]ethane-1-sulfonic acid (HEPES), pH=7.4) show very high stability when exposed to increasing voltage bias, with no membranes breaking when exposed to voltages of up to 500 mV, which is the highest voltage that can be applied with the instrumentation used. When n-butanol was present in the buffer, a decrease in membrane stability was observed, with membranes breaking when higher voltage biases were applied. The reversibility of the destabilization was shown by regenerating the high membrane stability by simply washing out the chaotropic solvent through repeated dilutions with the standard buffer solution. Results using butanol (BuOH) are shown in FIG. 10.

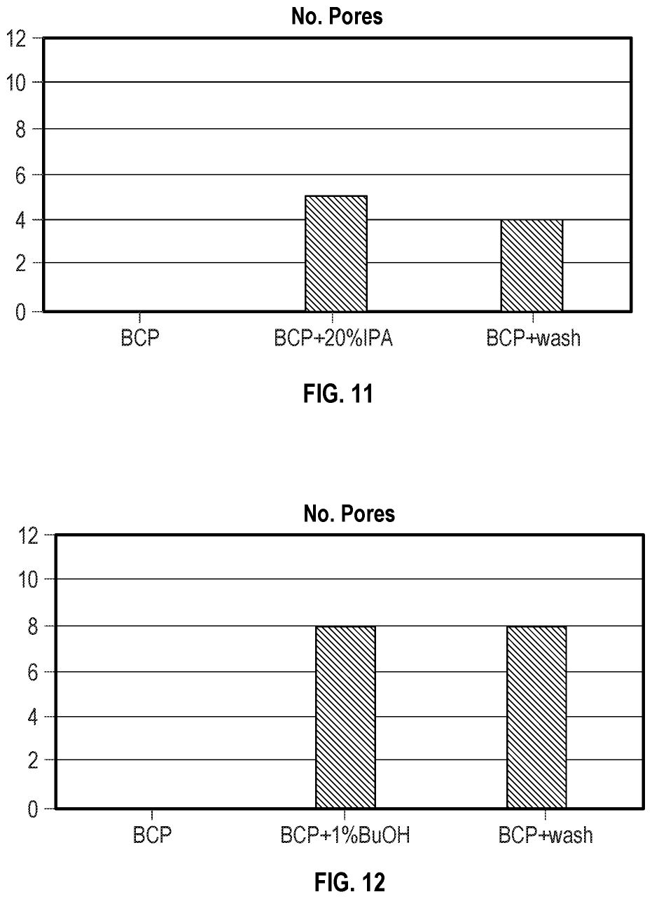

In line with the lower stability, nanopore insertion was possible when the membranes were subjected to electroporation in the presence of MspA nanopores. FIG. 11 is a plot showing the number of nanopores inserted into example membranes after washing in chaotropic solvent-containing buffer solutions. In this example, the chaotropic solvent was the amphiphilic solvent isopropyl alcohol. As shown, no nanopore insertion was possible when the membranes were subjected to electroporation in the presence of MspA nanopores. However, in a 20% isopropyl alcohol buffer solution, nanopores were able to be inserted. After washing the membranes repeatedly with the standard buffer solution, the nanopores largely remained inserted in the membrane.

FIG. 12 is a plot showing the number of nanopores inserted into example membranes after washing in chaotropic solvent-containing buffer solutions. In this example, the chaotropic solvent was the amphiphilic solvent n-butanol. As shown, no nanopore insertion was possible when the membranes were subjected to electroporation in the presence of MspA nanopores. However, in a 1% n-butanol buffer solution, nanopores were able to be inserted. After washing the membranes repeatedly with the standard buffer solution, the nanopores remained inserted in the membrane. As shown in FIGS. 11-12, not only are nanopores able to be inserted into the membrane in the presence of isopropyl alcohol or n-butanol, but the nanopores remain in the membrane after the nanopore-inserted membranes have the chaotropic solvent removed through repeated dilutions with the standard buffer solution.

Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of inserting a nanopore into a polymer membrane, the method comprising:
destabilizing the polymer membrane using a chaotropic solvent;
inserting the nanopore into the destabilized polymer membrane; and
removing the chaotropic solvent to stabilize the polymer membrane with the nanopore inserted therein.

2. The method of claim 1, wherein the chaotropic solvent comprises an amphiphilic solvent.

3. The method of claim 2, wherein the amphiphilic solvent comprises an alcohol, tetrahydrofuran, acetaldehyde, acetic acid, acetone, acetonitrile, ethylamine, or propanoic acid.

4. The method of claim 3, wherein the alcohol comprises isopropanol, n-butanol, ethanol, methanol, or 1-propanol.

5. The method of claim 2, wherein the amphiphilic solvent comprises a carbon chain with a length between 1 and 6 carbons.

6. The method of claim 2, wherein the amphiphilic solvent has a molar mass of less than about 75 grams per mole.

7. The method of claim 1, wherein the chaotropic solvent comprises a highly polar solvent.

8. The method of claim 7, wherein the highly polar solvent comprises a carbonyl group or a sulfonyl group.

9. The method of claim 7, wherein the highly polar solvent has a molar mass of less than about 80 grams per mole.

10. The method of claim 7, wherein the highly polar solvent comprises dimethyl sulfoxide, acetyl cyanide, urea, acetonitrile, formamide, dimethylformamide, methyl isocyanide, N-methyl-2-pyrrolidone, or triethylene glycol.

11. The method of claim 1, wherein the chaotropic solvent is removed through repeated dilutions using a buffer solution.

12. The method of claim 1, wherein the chaotropic solvent is removed through diffusion out of the polymer membrane.

13. The method of claim 1, wherein the nanopore is inserted into the destabilized polymer membrane using electroporation, pipette pump cycle, or detergent assisted nanopore insertion.

14. The method of claim 1, wherein the polymer membrane comprises molecules of a diblock copolymer, the molecules of the diblock copolymer comprising a hydrophobic block and a hydrophilic block coupled to the hydrophobic block.

15. The method of claim 14, wherein the polymer membrane comprises a first layer comprising a first plurality of molecules of the diblock copolymer, and a second layer comprising a second plurality of molecules of the diblock copolymer,
the hydrophilic blocks of the first plurality of molecules forming a first outer surface of the polymer membrane,
the hydrophilic blocks of the second plurality of molecules forming a second outer surface of the polymer membrane, and
the hydrophobic blocks of the first and second pluralities of molecules contacting one another within the polymer membrane.

16. The method of claim 14, wherein the chaotropic solvent destabilizes the polymer membrane by intercalating between the hydrophilic blocks.

17. The method of claim 14, wherein the chaotropic solvent destabilizes the polymer membrane by intercalating at interfaces between the hydrophilic blocks and the hydrophobic blocks.

18. The method of claim 1, wherein the polymer membrane comprises molecules of a triblock copolymer.

19. The method of claim 18, each molecule of the triblock copolymer comprising a hydrophilic block and first and second hydrophobic blocks, the hydrophilic block being coupled to and between the first and second hydrophobic blocks.

20. The method of claim 19, wherein the polymer membrane comprises a first layer comprising a first plurality of molecules of the triblock copolymer and a second layer comprising a second plurality of molecules of the triblock copolymer,
the hydrophilic blocks of the first plurality of molecules forming a first outer surface of the polymer membrane,
the hydrophilic blocks of the second plurality of molecules forming a second outer surface of the polymer membrane, and
the hydrophobic blocks of the first and second pluralities of molecules contacting one another within the polymer membrane.

21. The method of claim 19, wherein the chaotropic solvent destabilizes the polymer membrane by intercalating between the hydrophilic blocks.

22. The method of claim 19, wherein the chaotropic solvent destabilizes the polymer membrane by intercalating at interfaces between the hydrophilic blocks and the hydrophobic blocks.

23. The method of claim 18, each molecule of the triblock copolymer comprising a hydrophobic block and first and second hydrophilic blocks, the hydrophobic block being coupled to and between the first and second hydrophilic blocks.

24. The method of claim 23, wherein the polymer membrane comprises at least one layer comprising a plurality of molecules of the triblock copolymer,
the first hydrophilic blocks and the second hydrophilic blocks of the second plurality of molecules forming first and second outer surfaces of the polymer membrane.

25. The method of claim 23, wherein the chaotropic solvent destabilizes the polymer membrane by intercalating between the hydrophilic blocks.

26. The method of claim 23, wherein the chaotropic solvent destabilizes the polymer membrane by intercalating at interfaces between the hydrophilic blocks and the hydrophobic blocks.

27-28. (canceled)

29. A composition, comprising:
a polymer membrane; and
a chaotropic solvent destabilizing the polymer membrane.

30-56. (canceled)
